(19) United States
(12) Patent Application Publication
(10) Pub. No.: US 2025/0195547 A1
(43) Pub. Date: Jun. 19, 2025

(54) COMPOSITIONS AND METHODS OF TREATMENT FOR CONGENITAL DIARRHEAL DISORDER

(71) Applicant: VANDERBILT UNIVERSITY, Nashville, TN (US)

(72) Inventors: Izumi KAJI, Nashville, TN (US); James R. GOLDENRING, Nashville, TN (US)

(21) Appl. No.: 18/848,958

(22) PCT Filed: Apr. 18, 2023
(86) PCT No.: PCT/US2023/065887

§ 371 Date: Sep. 20, 2024

Related U.S. Application Data
(60) Provisional application No. 63/332,493, filed on Apr. 19, 2022.

(51) Int. Cl. A61K 31/662; A61K 45/06; A61P 1/00

(52) U.S. Cl. CPC A61K 31/662; A61K 45/06; A61P 1/00

(57) ABSTRACT

In one aspect, the disclosure relates to pharmaceutical compositions containing LPAR5 agonists including, but not limited to, compounds and methods for treating diseases and disorders associated with a mutation in Myosin Vb (MYO5B), decreased lysophosphatidic acid receptor 5 (LPAR5) expression, or both using the same. In one embodiment, the disclosed compositions and kits may be particularly useful in treating microvillus inclusion disease (MVID) and the diarrhea and malabsorption associated with the same. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 63/332,493, filed on Apr. 19, 2022, which is incorporated herein by reference in its entirety.

### STATEMENT REGARDING FEDERALLY SPONSORED RESEARCH OR DEVELOPMENT

This invention was made with government support under grants R01DK128190 and RC2 DK118640 awarded by the National Institute of Diabetes and Digestive and Kidney Diseases of the National Institutes of Health. The government has certain rights in the invention.

### BACKGROUND

Autosomal recessive Microvillus Inclusion Disease (MVID) is characterized by feeding-induced dehydrating diarrhea usually starting in the first week after birth (Cutz et al., 1989; Davidson et al., 1978). MVID is caused by homozygous or compound heterozygous inactivating mutations in Myosin Vb (MYO5B), an essential motor protein in the intestinal epithelial cells (Erickson et al., 2008; Muller et al., 2008; Ruemmele et al., 2006) that regulates apical vesicle trafficking among other functions. Diarrhea in MVID patients is associated with villus blunting, loss of microvilli on the apical membrane, the accumulation of large autophagic lysosomes, and loss of apical transporters, such as sodium/proton exchanger 3 (NHE3) and sodium-dependent glucose transporter 1 (SGLT1) (Thiagarajah et al., 2018) (FIG. 9). To treat the severe and life-threatening malabsorption syndrome in MVID patients to date, continuous total parental nutrition therapy or intestinal transplantation are the only options (Bowman et al., 2022).

Several experimental models of MVID have been established to investigate drug treatment options, including MYO5B-deficient mouse strains (Weis et al., 2016), MVID patient-mimicking mice with a compound heterozygous mutation at Myo5b(G519R) (Burman et al., 2022), and a genetically engineered pig model with a homozygous MYO5B(P663L) mutation that is homologous with the mutation observed in Navajo MVID patients (Engevik et al., 2020). It was recently found that a bioactive phospholipid, lysophosphatidic acid (LPA), can promote microvillus structure and improve sodium absorption in MYO5B-deficient mice (Kaji et al., 2020). LPA treatment also normalized the differentiation of tuft cell lineage in the MYO5B-deficient intestinal mucosa that correlate with epithelial cell differentiation and maturation (Kaji et al., 2021). These findings suggest that the trafficking and differentiation blockades induced by MYO5B inactivation can be overcome by LPA-mediated pathways, leading to the possible development of treatments for MVID that could obviate the need for intestinal transplantation or long-term total parental nutrition.

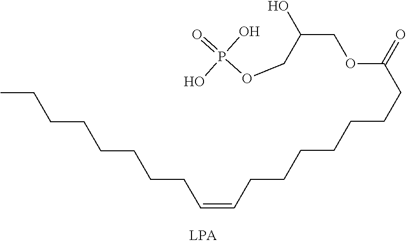 [SMILES: CCCCCCCC/C=C\CCCCCCCC(=O)OCC(O)COP(=O)(O)O]

Although the topology of each receptor protein has not been clarified, the abundant expressions of LPA receptor (Lpar) family members, Lpar1, Lpar5, and Lpar6 are identified in the intestinal epithelial cells (Haber et al., 2017; Li et al., 2005). Recent studies utilizing mouse enteroids suggest that LPAR activations are important for epithelial proliferation. LPA supplementation to the medium enhances the proliferation and differentiation of enteroids through LPAR1 independent of epidermal growth factor (Konno et al., 2019). Genetically engineered mouse enteroids also demonstrated that LPAR2 and LPAR5 reciprocally support cell proliferation (Liang and Yun, 2022). Furthermore, LPAR5 depletion mouse models revealed that epithelial and non-epithelial LPAR5 signaling is critical for intestinal stem cell proliferation (Liang et al., 2022). Previous studies have demonstrated that LPAR5 activation stimulates the membrane trafficking of NHE3 in enterocytes that facilitate sodium and water absorption (Jenkin et al., 2018; Lin et al., 2010).

Based on those previous reports, it is hypothesized that LPAR5 mostly mediates the therapeutic effect of LPA treatment that were seen in the MYO5B deficient mouse and enteroid models. In addition, the natural LPA is not an ideal drug to obtain consistent dosing because of its poor solubility and stability. Several LPAR subtype-specific agonists and antagonists have been developed in terms of treatment for different diseases (Liu et al., 2021; Meduri et al., 2021), however, LPAR5 activator has not been tested on congenital diarrheal diseases.

Despite advances in MVID research, there is still a scarcity of compounds that are potent and effective treatments for MVID and that are also soluble and stable at physiological conditions in order to provide effective treatment at a consistent dosage to patients in need thereof. These needs and other needs are satisfied by the present disclosure.

### SUMMARY

In accordance with the purpose(s) of the present disclosure, as embodied and broadly described herein, the disclosure, in one aspect, relates to pharmaceutical compositions containing LPAR5 agonists including, but not limited to, compounds having Formula I, kits comprising the same, and methods of treating diseases and disorders associated with a mutation in Myosin Vb (MYO5B), decreased lysophosphatidic acid receptor 5 (LPAR5) expression, or both using the same.

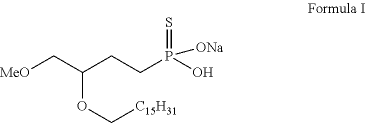

In one embodiment, the disclosed compositions and kits may be particularly useful in treating microvillus inclusion disease (MVID) and the diarrhea and malabsorption associated with the same.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1B shows the impact of LPA treatment on epithelial cellular metabolic pathways in mice. FIG. 1A: Transcriptional analysis in intestinal epithelial cells indicates significantly altered energy metabolic pathways in MYO5B deficient (Myo5bΔINT) mice compared to control mice. LPA treatment on Myo5bΔINT mice significantly increased Hmgcs2, a rate-limiting ketogenic enzyme, compared to non-treated MYO5B deficient mice. FIG. 1B: Daily LPA treatment for Myo5bΔINT mice increases the expression of a ketogenic enzyme, HMGCS2, in the jejunal epithelial cells.

FIGS. 2A-2E show Compound-1 treatment for Myo5bΔINT mouse intestine mimicked the effect of LPA. FIG. 2A: Compound-1 structure and experimental design of mouse treatment. FIG. 2B: Villus/crypt ratio in Myo5bINT mouse jejunum. Bars indicate median values and each datapoint represents an average value of 10 regions in each mouse (N=4-5 mice per each genotype). *P<0.05 by t-test. FIG. 2C: In vehicle-treated Myo5bΔINT mouse intestine, NHE3 is diffuse in cytoplasm of epithelial cells and some enterocytes lack villin. Compound-1-treated mice demonstrate the colocalization of NHE3 with villin on the brush border. FIG. 2D: Immunostaining for SGLT1 in Myo5bΔINT mouse jejunum with or without Compound-1 treatment. FIG. 2E: Immunostaining for HMGCS2 in Myo5bΔINT mice that received Compound-1 daily for 4 days demonstrates robust expression of HMGCS2 in most epithelial cells in jejunum. Scale bar=50 μm.

FIGS. 3A-3E show the effect of LPAR agonists on tuft cell differentiation. FIG. 3A: Immunostained jejunum from Myo5bΔINT mice that were treated with Compound-1 or vehicle. DCLK1+ tuft cells do not express the proliferative marker, Ki67. Actg1 (white) signals demonstrate villus morphology and microvillus inclusions. FIG. 3B: Tuft cell population was determined in the jejunum of mice that received one of LPAR agonists. (N=2-6 mice). *P<0.05 by ANOVA with Tukey's comparisons. FIG. 3C: Enteroids were generated from jejunal crypts of wild-type (WT) and genetically engineered pig with a point mutation at MYO5B(P663L) and expanded in Human Organoid Growth Medium. Whole well images were analyzed to determine organoid forming efficacy and perimeters in wild type (WT) and MVID model with a point mutation at MYO5B(P663L). Scale=100 μm. *P<0.05 by two-way ANOVA. FIG. 3D: Whole-mount immunostaining for tuft cell markers, pY1798-Girdin and POU2F3, and phalloidin staining for F-actin. FIG. 3E: Compound-1 (100 nM) or vehicle was supplemented to differentiation medium for 5 days. MYO5B(P663L) enteroids have less tuft cells compared to WT, and Compound-1 treatment significantly increased tuft cell differentiation. Each datapoint indicates each enteroid. ***P<0.001 by ANOVA with Dunnett's multiple comparisons.

FIGS. 4A-4B show cell-autonomous effects of MYO5B loss and Compound-1 treatment. FIG. 4A: Selected transcription signatures in enteroids generated from Myo5bΔINT and Myo5b(G519R) mice. FIG. 4B: MYO5B deficient enteroids were treated with Compound-1 (100 nM) or vehicle for 2 days. Group 1 genes are significantly increased by MYO5B loss and decreased by Compound-1 treatment. Only Nox1 is decreased by MYO5B loss and reversed by Compound-1, while Group 3 genes are increased by MYO5B loss and further increased by Compound-1. N=4 mice per each genotype.

FIG. 5 shows a schematic of how Compound-1 (LPAR5 agonist) can ameliorate MYO5B deficient intestine according to one exemplary embodiment. Trophic effect of Compound-1 on MYO5B deficient mouse intestine is likely based on altered cellular metabolic pathway and progenitor cell differentiation. Epithelial cell differentiation toward mature enterocytes and tuft cells are important to increase absorptive surface area to improve nutrient absorption.

FIG. 6 shows relative expression of LPA receptor (LPAR) subtypes in intestinal epithelial cells isolated from control and MYO5B deficient mice. Mean±SEM. **P<0.01, ***P<0.001 by ANOVA with LSD test. N=3 mice per group.

FIG. 7 shows mitochondrial structures are disrupted by functional MYO5B loss in villus and crypt cells.

FIG. 8 shows a summary of differential expression genes in mouse enteroids. Four individual cultures from four mice of each group are analyzed; iKO represents Myo5bΔINT, iKO_C1 is Compound-1-treated Myo5bΔINT, G519R is Myo5b(G519R), and Control is healthy littermates.

FIG. 9 shows micrographs of intestinal cells in normal patients compared to patients with MYO5B (G519R) mutations.

FIGS. 10A-10B show Compound-1 increased tuft cell population in pig enteroids. Enteroids were generated from the jejunum of wild type or MVID model pigs with a point mutation of MYO5B(P663L). Compound-1 (100 nM) or vehicle (PBS) were added into differentiation medium.

FIGS. 11A-11B show differential sensory tuft cell differentiation in EpCAM KO mice, a model of congenital tufting enteropathy. FIG. 11A shows controls while FIG. 11B shows imaging of KO mice. FIGS. 11C-11D show decreased apical expression of SGLT1 in EpCAM KO mice. FIG. 11C shows controls while FIG. 11D shows imaging of KO mice. Scale bar is 50 μm. These images indicate subjects with congenital tufting enteropathy have similar intestinal failure to subjects with MVID.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Embodiments of the present disclosure provide for compositions, pharmaceutical compositions, and methods of treating congenital diarrheal disorder. In an aspect, the composition and pharmaceutical composition can include an effective amount or therapeutically effective amount of compound-1 and derivatives thereof to a subject in need to treat congenital diarrheal disorder. In addition, the present disclosure includes administering an effective amount or therapeutically effective amount of compound-1 and derivatives thereof to a subject in need to treat congenital diarrheal disorder.

Using a mouse model of microvillus inclusion disease (MVID), it has been reported that the administration of lysophosphatidic acid (LPA) can ameliorate epithelial cell differentiation and nutrient absorption (Kaji et al. Gastroenterology 2020). To maximize the therapeutic effect of LPA, a selective agonist for LPA receptor subtype 5 (LPAR5) has been synthesized, namely Compound-1 (C₂₀H₄₂NaO₅PS, mw=448.57) at Vanderbilt Chemical Synthesis Core. The Compound-1 synthetic pathway and LPAR5 affinity have been reported by Jiang et al. (Bioorganic & Medicinal Chemistry Letters 2013), but the effect of Compound-1 treatment on congenital diarrhea diseases have not been investigated. Here, it is shown that LPAR5 expression is significantly higher than other LPAR subtypes in both control and MYO5B-deficient (MVID model) mouse intestines. Comparing to vehicle-treated mice, intraperitoneal injection of Compound-1 significantly improved villus/crypt ratio. Nutrient transporters of mature enterocytes, such as NHE3 and SGLT1 are mislocalized from apical membrane in MVID mouse intestine and their localization are partially recovered to the apical membranes by Compound-1 treatment. Furthermore, the decrease in differentiated tuft cell numbers in MVID mice with MYO5B loss is significantly increased by Compound-1 treatment, suggesting that LPAR5 activation can ameliorate proper cell differentiation in MVID model mice. Compound-1 as a sodium salt is highly soluble in water and it would be an useful drug treatment for malabsorption in congenital diarrheal patients, including but not limited to MVID.

In some aspects, in addition to MYO5B, mutations, other mutations can cause variant MVID or MVID-like conditions. In an aspect, a loss in syntaxin 3 function causes variant MVID. In another aspect, a UNC45A deficiency causes an MVID-like phenotype by impairing MYO5B-dependent apical trafficking. In any of these aspects, the disclosed compositions and methods of treatment can be used to treat malabsorption in patients having these mutations.

These findings suggest that LPA and LPAR5 agonists can reverse mislocalization of transporters in enterocytes that leads to life threatening diarrhea in MVID patients. These findings indicate that treatment of MVID patients with Compound-1 or derivatives of Compound-1 could provide relief from diarrhea and provide effective treatment for MVID patients allowing them to come off total parenteral nutrition and avoid the need for intestine/liver transplantation.

### Method of Treating or Preventing a Disease

In one aspect, disclosed herein is a method of treating or preventing a disease in a subject, wherein the disease is associated with a mutation in Myosin Vb (MYO5B), a mutation in UNC45A, a mutation in syntaxin 3, decreased lysophosphatidic acid receptor 5 (LPAR5) expression, or both, and wherein the method includes at least the step of administering a composition comprising a therapeutically effective amount of an LPAR5 agonist to the subject. In a further aspect, the LPAR5 agonist can include one or more of the following: lysophosphatidic acid, [(2S)-2-hydroxy-3-octadecoxypropyl] dihydrogen phosphate, [(2Z,6Z)-3,7,11-trimethyldodeca-2,6,10-trienyl] dihydrogen phosphate, farnesyl diphosphate, octyl thiophosphatidic acid, or N-arachidonoylglycine, or can be a compound having Formula I or a derivative thereof.

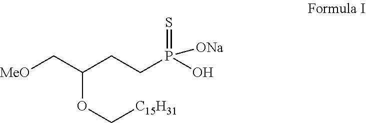

In another aspect, the subject can be a mammal such as, for example, a human, pig, mouse, rat, dog, cat, or rabbit, or any other mammal experiencing a pathology involving a MYO5B mutation and/or decreased LPAR5 expression.

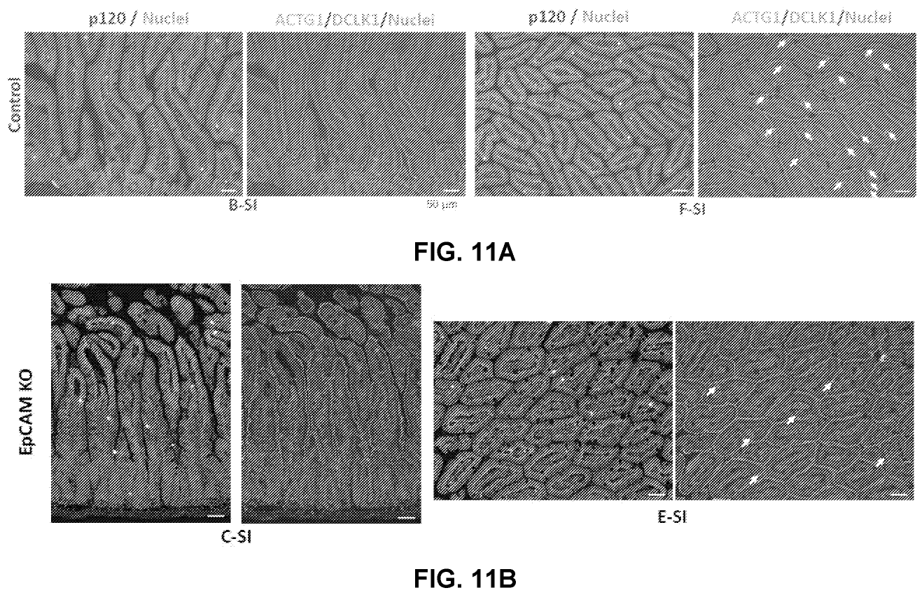

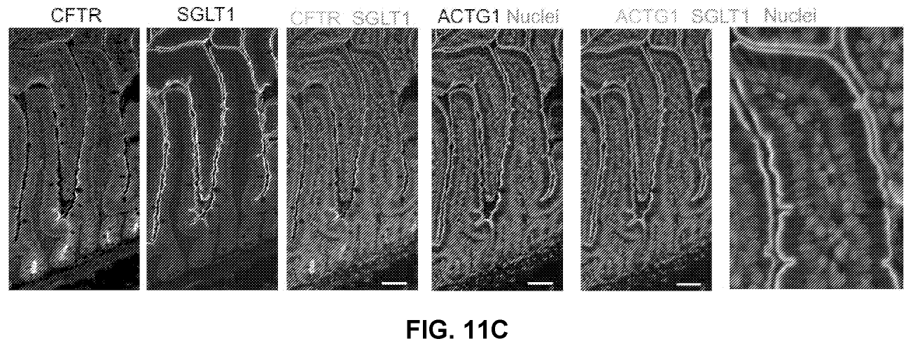

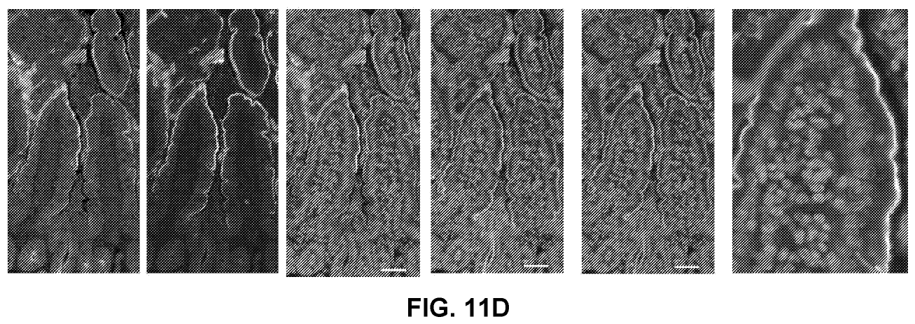

The compounds and compositions disclosed herein may be particularly useful, in one aspect, for treating microvillus inclusion disease (MVID), cholestatic liver disease, diacylglycerol-acyltransferase 1 (DGAT1) deficiency, congenital or other tufting enteropathy, variant MVID, an MVID-like phenotype, and/or combinations thereof. Without wishing to be bound by theory, subjects having congenital tufting enteropathy (CTE) may display tuft cell differentiation deficits through Notch signaling abnormality, similar to subjects having MYO5B deficiencies (see FIGS. 11A-11D). In a further aspect, this indicates LPAR5 agonists may be therapeutic for CTE as well.

As used herein, the term “effective amount” refers to an amount that is sufficient to achieve the desired modification of a physical property of the composition or material. For example, an “effective amount” of a pharmaceutical composition refers to an amount that is sufficient to achieve the desired improvement in the property modulated by the formulation component, e.g., achieving the desired level of treatment. The specific level in terms of wt % in a composition required as an effective amount will depend upon a variety of factors including the amount and type of pharmaceutical composition, the subject, severity of illness, and the like.

In a further aspect, the MYO5B mutation can be any type of mutation and/or can produce any type of ineffective or otherwise nonfunctional protein. In one aspect, the mutation can be an insertion or deletion mutation and can produce a truncated or a misfolded protein, or any combination thereof. In still another aspect, the mutation can be homozygous or compound heterozygous.

In any of these aspects, the disclosed compositions can include any useful carriers and/or excipients as described herein.

In an aspect, treating a disease can reduce the severity of at least one symptom of the disease in the subject as compared to the severity of the same symptom prior to initiating treatment. In a further aspect, the at least one symptom can be diarrhea or malabsorption. In some aspects, treating the disease causes full recovery. In other aspects, treating the disease causes at least partial recovery (i.e., at least 50% recovery, at least 75% recovery, or at least 90% recovery) such that other treatments may be discontinued.

In one aspect, administering the composition includes a subcutaneous method of administration or intravenous administration. In some aspects, the composition can be one or more times per day for 14 or more days or for one, two, three, four, five, or six or more months as needed, or for 1, 2, 3, 4, or 5 or more years. In another aspect, the composition can be administered at a higher initial dosage and/or more frequently to treat acute initial stages of the disease, and the dosage can be tapered or decreased as the subject stabilizes. In some aspects, the dosage form can be altered after initial treatment (i.e., a patient can start by receiving intravenous administration in a medical facility and, after stabilization, can be transferred home with a maintenance dosage form that can be administered by the subject or a caregiver such as, for example, an oral dosage form). In some aspects, subjects may have different grades of symptoms based on different MYO5B or other mutations. Further in this aspect, different routes of administration may be possible—for example, a patient with mild symptoms may be able to begin treatment with an oral administration route.

In some aspects, the composition can be administered at a dosage of from about 5 mg to about 10 mg per kg of body weight of the subject, or from about 5 to about 8 mg, or from about 6 to about 7.5 mg per kg of body weight of the subject.

In a further aspect, in some cases, particularly with patients having severe disease, at least one additional treatment may be administered to the subject, especially during the initial stages of treatment. In one example, a patient beginning a treatment regimen can simultaneously receive total parenteral nutrition. Further in this aspect, when the patient stabilizes, total parenteral nutrition can be decreased or discontinued and the patient reintroduced to standard oral feeding.

Also disclosed herein are kits for treating a disorder associated with a mutation in Myosin Vb (MYO5B), decreased lysophosphatidic acid receptor 5 (LPAR5) expression, or both in a subject, the kits including at least: (a) a therapeutically effective amount of a compound of Formula I and at least one pharmaceutically acceptable excipient; and (b) instructions for using the kit.

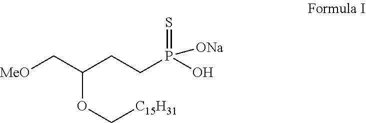

In one aspect, the compound of Formula I and the at least one pharmaceutically acceptable excipient can be packaged separately, such as, for example, when the compound of Formula I is provided as a lyophilized powder to be resuspended in an injectable solution by a medical provider prior to administering to the patient. Further in this aspect, the therapeutically effective amount of the compound in the kit can include a unit dosage of from about 12.5 mg to about 1000 mg, from about 12.5 mg to about 50 mg, from about 50 mg to about 250 mg, from about 250 mg to about 500 mg, or from about 500 mg to about 100 mg. In some aspects, separate packaging of the compound of Formula I and the excipient can prolong the shelf life of the compound. In another aspect, the kit can contain a single dosage of the compound or multiple dosages of the compound (i.e., an injectable solution where amount injected can be varied based on patient's body weight).

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a mutation,” “a drug,” or “an additional treatment,” includes, but is not limited to, mixtures or combinations of two or more such mutations, drugs, additional treatments, and the like

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a drug such as, for example, Compound-1 refers to an amount that is sufficient to achieve the desired improvement in the property modulated by the formulation component, e.g. achieving the desired level of improvement in intestinal absorption. The specific level in terms of wt % in a composition required as an effective amount will depend upon a variety of factors including the severity of the condition being treated, amount and type of additional or auxiliary treatments being administered, method of administration, and particular MYO5B mutation the subject carries.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a bird, reptile, amphibian, mammal (e.g. human, canine, feline, equine, cattle, etc.). In an aspect, the subject is a human. In an aspect, the subject is a domesticated animal such as a dog or cat. “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as infections and consequences thereof. The effect can be therapeutic in terms of a partial or complete cure of a disease (e.g., congenital diarrheal disorder), condition, symptom or adverse effect attributed to the disease, disorder, or condition. The term “treatment” as used herein can include any treatment of congenital diarrheal disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration. In an aspect, the dosage for administering to a subject (e.g., a mammal, specifically a human) each active agent the present disclosure, where dosage can be adjusted as needed based on the active agent, the type of subject, the condition of the subject, the state of the disease or condition (e.g., congenital diarrheal disorder), and the like.

As used herein, “therapeutic” can refer to treating, healing, and/or ameliorating a disease, disorder, condition, or side effect, or to decreasing in the rate of advancement of a disease, disorder, condition, or side effect.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder (e.g., congenital diarrheal disorder) being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

A response to a therapeutically effective dose of a disclosed compound (e.g., active agent) and/or pharmaceutical composition (e.g., including the active agent), for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia.

The disclosed composition (e.g., pharmaceutical composition including the active agent) or active agent can include those suitable for oral, rectal, topical, pulmonary, nasal, and parenteral administration (e.g., and in particular, rectal, topical, pulmonary, nasal, and parenteral administration), although the most suitable route in any given case will depend on the particular subject, and nature and severity of the conditions for which the active agent is being administered. In a further aspect, the disclosed composition (e.g., pharmaceutical composition) or agent can be formulated to allow administration orally, nasally, via inhalation, parenterally, transmucosally, transdermally, intramuscularly, intravenously, intradermally, subcutaneously, intraperitoneally, intraventricularly, intracranially and intratumorally (e.g., and in particular, nasally, via inhalation, parenterally, transmucosally, transdermally, intramuscularly, intravenously, intradermally, subcutaneously, intraperitoneally, intraventricularly, intracranially and intratumorally).

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, metabolites, esters, amides, salts of esters or amides, and N-oxides of a parent compound. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Unless otherwise specified, temperatures referred to herein are based on atmospheric pressure (i.e. one atmosphere).

### Kits

The disclosed compounds and/or pharmaceutical compositions comprising the disclosed LPAR5 agonists can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

In a further aspect, the present disclosure also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed compositions and/or the disclosed methods of using or treating.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Results

### LPA Treatment Alters MYO5B Loss-Induced Cellular Metabolic Pathways

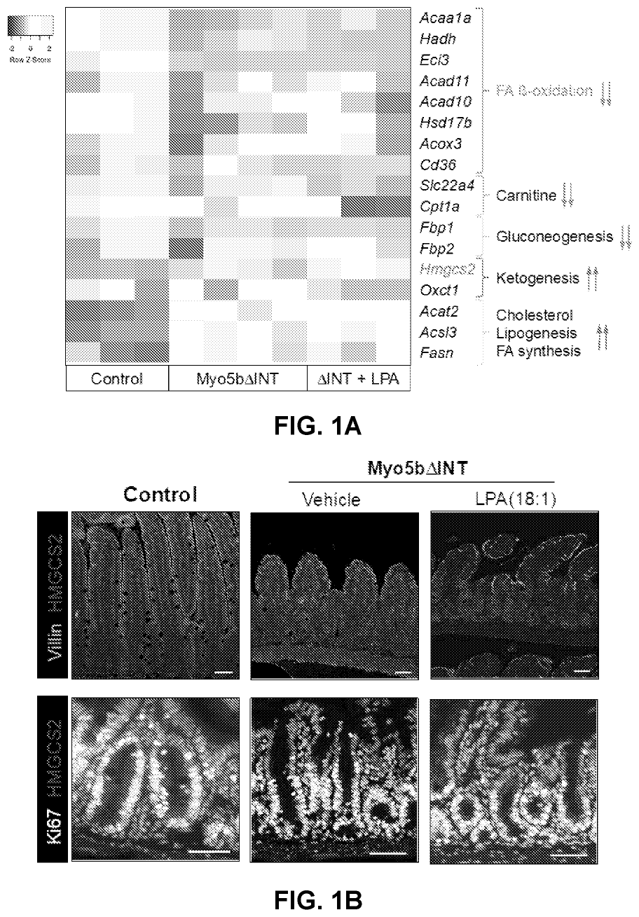

Epithelial cell differentiation pathway closely interacts with epithelial cellular metabolism (Cheng et al., 2019). Utilizing the RNA-seq data of jejunal epithelial cells from Myo5bΔINT mice, significant alterations in cellular metabolic pathways induced by MYO5B loss (GSE139302) have been identified (FIG. 1A). Gene expressions that mediate fatty acid oxidation and carnitine signaling are significantly downregulated that are essential for stem cell maintenance (Chen et al., 2019; Stine et al., 2019). Instead, MYO5B loss significantly upregulated genes that mediate fatty acid synthesis and lipogenesis. In vivo treatment with LPA (18:1) did not reverse fatty acid oxidation or lipogenesis pathways. Interestingly, LPA treatment significantly upregulated transcription of Hmgcs2, a rate-limiting ketogenic enzyme, compared to vehicle-treated Myo5bΔINT or healthy littermate (tamoxifen-untreated) mice (FIG. 1A). The protein expression of Hmgcs2 in healthy mouse jejunum is limited to the stem cells, and ketone bodies are important for in stem cell function and differentiation of secretory cells (Cheng et al., 2019). MYO5B-deficient intestine showed expanded Hmgcs2 expression in the proliferative cell zone, and systemic LPA treatment further increased the expression of Hmgcs2 in both proliferating and differentiating epithelial cells (FIG. 1B). These observations suggest that MYO5B loss induces a starvation-like phenotype in proliferative epithelial cells and that LPA receptor activation increased ketone body production as alternative energy metabolic fuel, resulting in the promotion of epithelial cell differentiation.

### MYO5B Loss Decreases LPAR Expressions in Intestinal Epithelial Cells

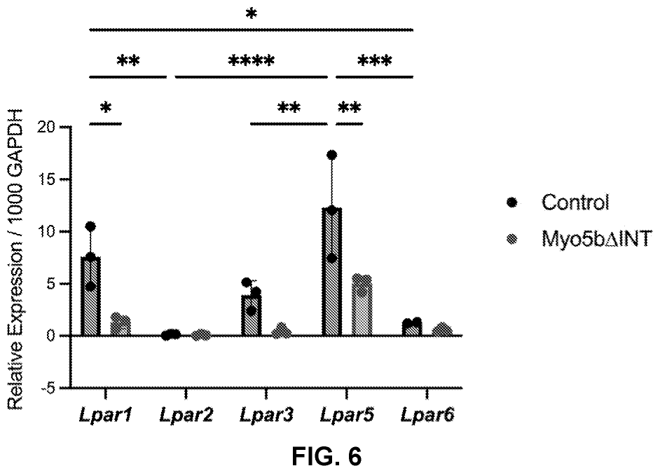

Using real-time PCR, mRNA expressions were investigated in jejunal epithelial cells of tamoxifen-induced Myo5bΔINT and control mice. Among LPAR1-6, LPAR4 was undetectable level and all LPAR subtypes were significantly lower in Myo5bΔINT mice than in control (FIG. 6). LPAR5 expression was still prominent in Myo5b-deficient intestine.

### Compound-1 Represents Trophic Effect of Natural LPA

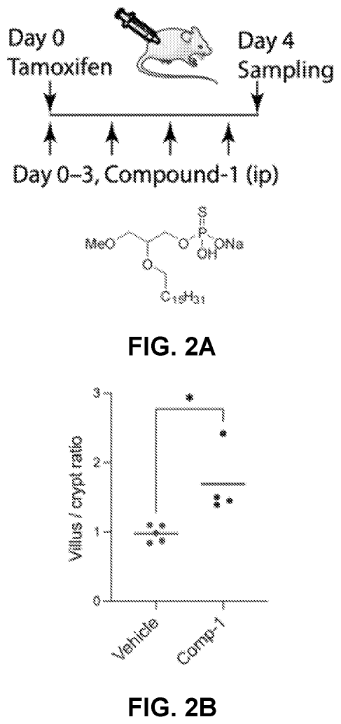

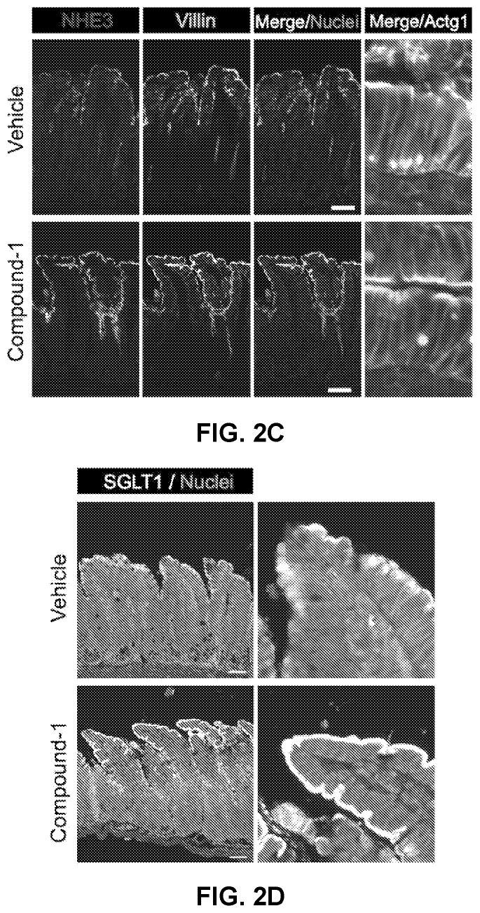

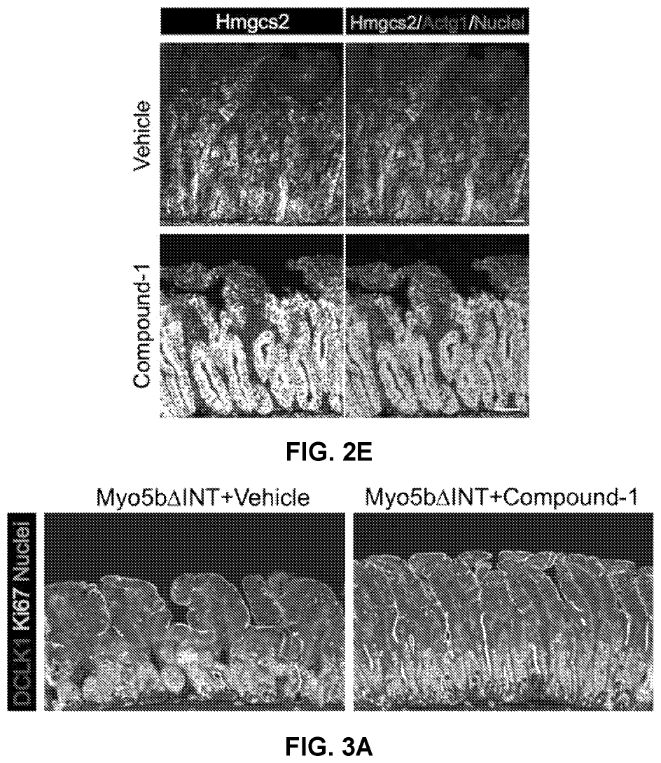

The effects of synthetic agonist for LPA receptor 5 (LPAR5), Compound-1, was investigated on adult tamoxifen-induced Villin-Cre^{ERT2}; Myo5b^{flox/flox} (Myo5bΔINT) mice. Intestinal villus/crypt ratio was significantly improved by daily Compound-1 (1 mg/kg) treatment, indicating the increase in nutrient absorptive area (FIGS. 2A-2B). MYO5B deficient intestines lose the apical localization of important sodium transporters, such as Na+/H+ exchanger 3 (NHE3) and sodium-dependent glucose transporter 1 (SGLT1). Those transporter localizations were partially reversed to normal in Compound-1-treated Myo5bΔINT mouse small intestine, suggesting that Compound-1 enhances functional brush border formation similarly to natural LPA treatment (FIGS. 2C-2D). Compound-1 treatment on Myo5bΔINT mice, once daily by intraperitoneal injection, remarkably upregulated Hmgcs2 expression in the most epithelial cells (FIG. 2E).

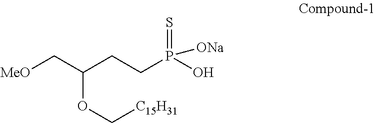

### MYO5B Loss Alters Mitochondrial Structures in Epithelial Cells

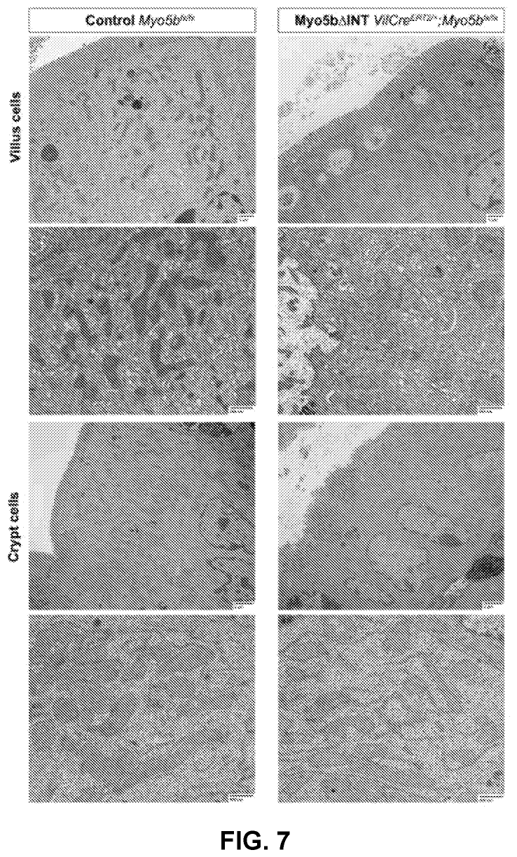

Transmission electron microscopy was utilized to investigate mitochondrial morphologies in Myo5bΔINT and healthy littermate control mouse jejunum. As shown in (FIG. 7), control enterocytes in villi had long uniformed microvilli with approximately 2 μm length, while crypt epithelial cells possessed shorter microvilli. The subapical space contained numbers of mitochondria with dense and organized crista structures. Crypt epithelial cells had large and round-shaped mitochondria compared to villus cells, implicating that mitochondria have different functions between different cell types depending on energy requirement. Crypt cells of MVID model animals or MVID patient tissues have not been precisely described in TEM level. Myo5bΔINT mouse jejunum showed immature microvilli and accumulation of abnormal vesicles in subapical space both in villi and crypts (FIG. 7), similarly to that in the MVID patient-mimicking model, Myo5b(G519R) mice (Burman and Momoh et al., 2023, CMGH). In Myo5bΔINT tissues, mitochondria in remained subapical area were swelled and lost organized crista structures. These observations are consistent with the MYO5B loss-induced disruption of energy metabolic pathways.

### Compound-1 Treatment Ameliorates Tuft Cell Differentiation

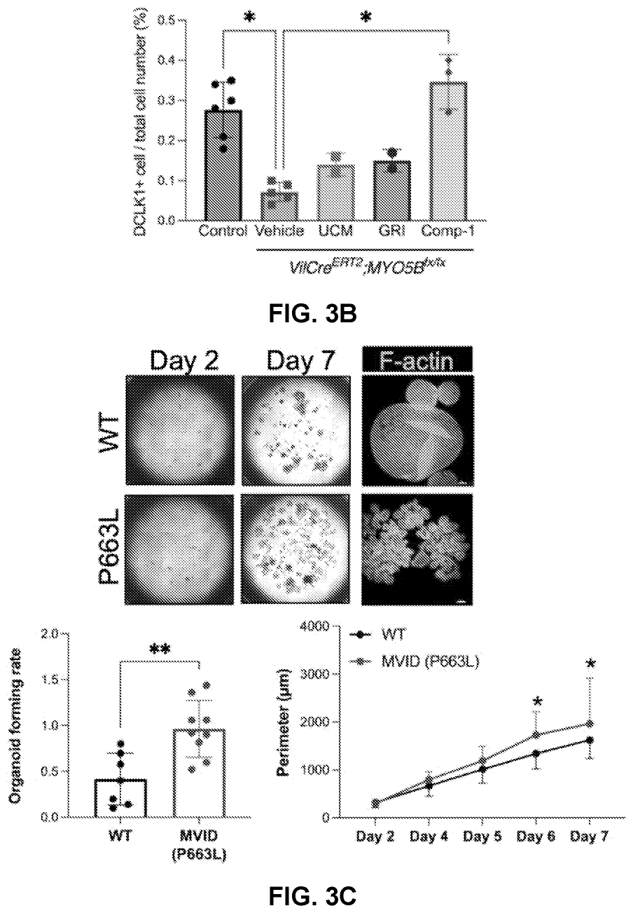

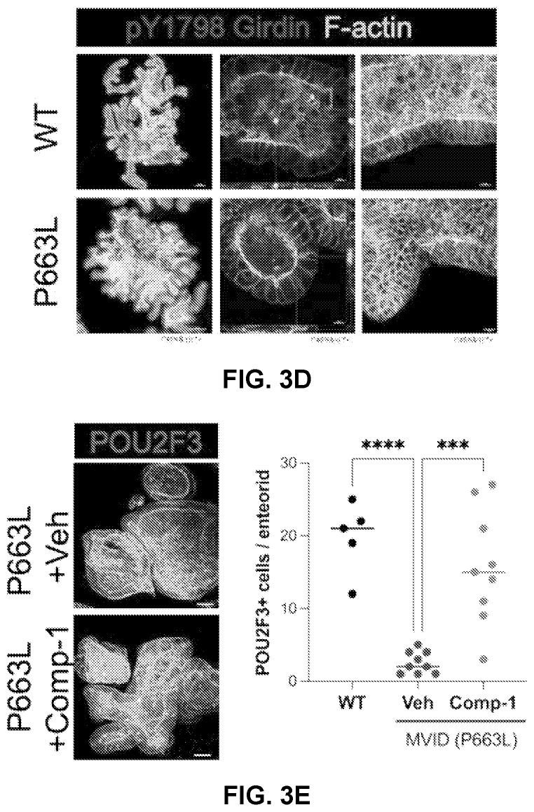

It was recently reported that functional MYO5B loss disrupts epithelial cell lineage differentiation through imbalance of epithelial Wnt/Notch signaling (Kaji et al. 2021 JCI Insight). To assess the epithelial cell differentiation capability, the population of sensory epithelial cell, tuft cell, was measured in Myo5bΔINT mice with LPAR agonist treatment. Similarly to natural LPA (18:1, 3 mg/kg), Compound-1 (1 mg/kg) reversed tuft cell differentiation in the Myo5bΔINT intestine (FIGS. 3A-3B). Other synthetic LPAR agonists had no significant effect, including UCM-05194 (10 mg/kg) or GRI977143 (3 mg/kg), selective for LPAR1 or LPAR2, respectively.

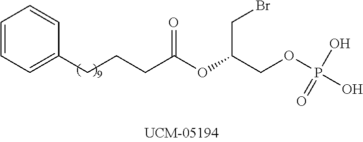

Compound-1-induced tuft cell differentiation was tested in pig enteroid cultures that possess MVID patient-mimicking mutation at MYO5B(P663L) (orthologue of human P660L) (Engevik 2020). The MVID model enteroids that were generated from pig jejunum with MYO5B(P663L) mutation showed higher enteroid forming rate than wild type (WT) jejunal enteroids (FIG. 3C). Pig tuft cells were differentiated in ODM for 6 days and immunostained for phosphorylated girdin (pY-1798) and the key transcription factor for sensory cell differentiation, POU2F3. Compared to WT pig enteroids, MYO5B(P663L) enteroids had significantly less POU2F3+ cells in each enteroid (FIGS. 3D-3E). These results suggest that MYO5B loss-induced differentiation deficits are not Myo5b-deficient mouse model specific. Daily supplementation of Compound-1 (100 nM) into the ODM significantly increased tuft cell numbers in MYO5B(P663L) enteroids, suggesting that LPAR5 activation has cell-autonomous effects on MVID patient-mimicking enteroids (FIG. 3E).

### Compound-1 Treatment Alters Transcription Signatures in MYO5B Deficient Enteroids

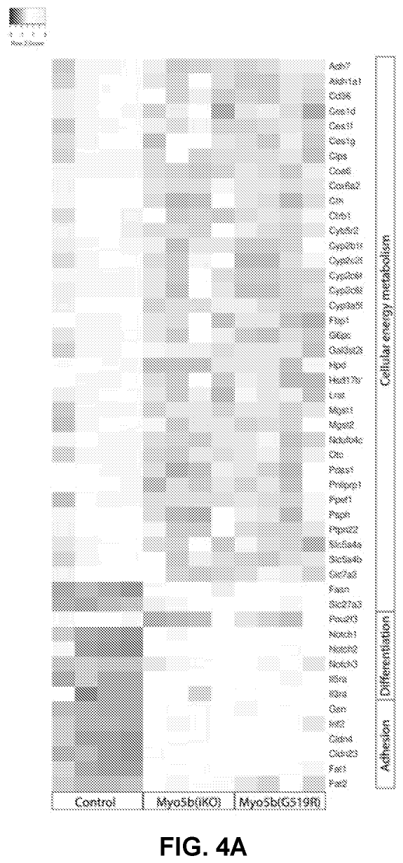

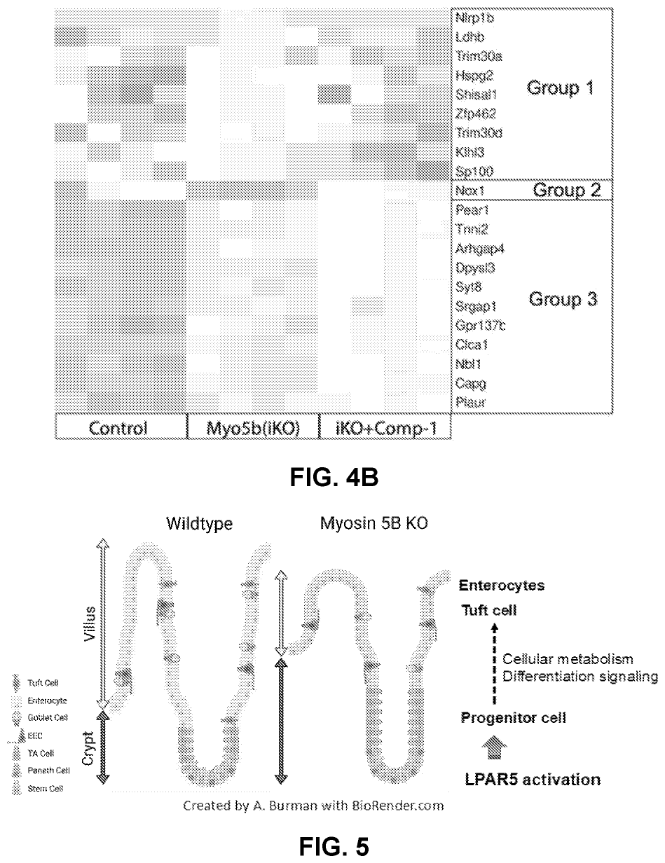

To assess cell-autonomous effect of MYO5B loss and Compound-1 treatment in epithelial cells, bulk transcriptional analysis was performed in enteroids that were generated from Myo5bΔINT and Myo5b(G519R) mouse jejunum. The overall alteration pattern of gene expression was quite similar between Myo5bΔINT and Myo5b(G519R), consistent with the findings in in vivo mouse tissues (Burman et al., 2022). Some Myo5bΔINT enteroids were treated with Compound-1 (100 nM) for 2 days during differentiation to seek direct LPAR5 targets in enterocytes. Lpar2, Lpar5, and Lpar6 transcriptions were detected in all samples and had no significant difference. Although Myo5bΔINT and Myo5b(G519R) enteroids grew in similar manner to control enteroids, numerous energy metabolic enzymes and 20 mitochondrial genes were significantly downregulated by functional loss of MYO5B (FIG. 4A). Similar to the transcriptional signature in epithelial cells from tissues, Fasn and Slc27a3 were upregulated that indicate fatty acid synthesis (FIG. 4A). In contrast, Hmgcs2 expression was comparable between control and MYO5B deficient enteroids with and without Compound-1, suggesting that ketogenic pathway is maintained in enteroid culture condition.

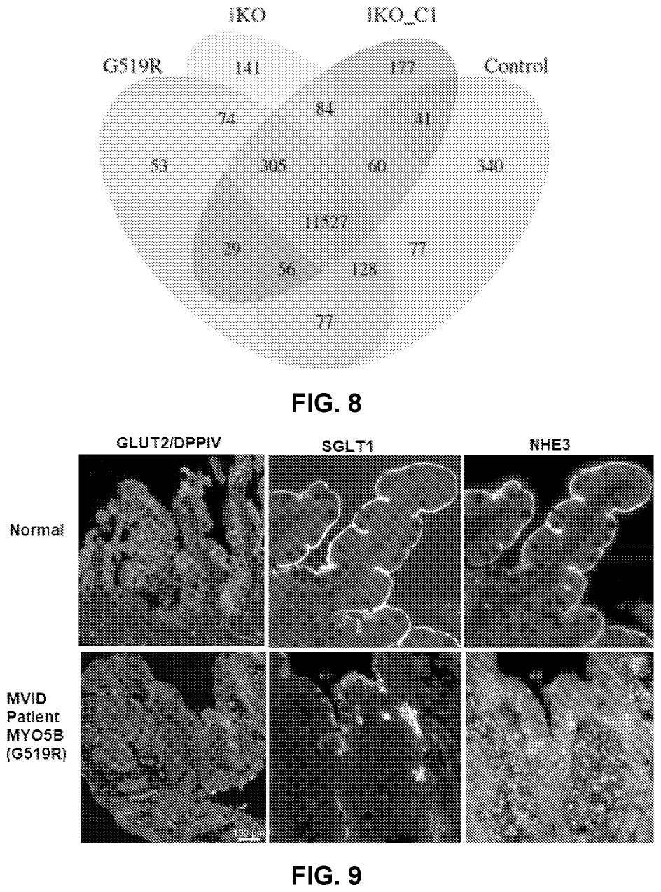

Both Myo5bΔINT and Myo5b(G519R) enteroids had significantly downregulated Pou2f3 expression and upregulated Notch1, Notch2, Notch3, Jag1, and Maml2, indicating that tuft cell lineage differentiation was blocked likely through abnormally enhanced Notch signaling. Highly upregulated genes by functional MYO5B loss were implicated in inflammatory response and abnormal cell-cell adhesion, including Mcam, Fat1, Fat2, Inf2, Col4a, Col4a2, Cldn4, and Cldn23, which were very low or undetectable levels in healthy enteroids (FIG. 4A). In vitro Compound-1 treatment significantly reversed several inflammation- and infection-related genes, such as Nlrp1b, Ldhb, Hspg2, and Trim30, suggesting that Compound-1 directly reversed some epithelial cell deficits induced by MYO5B loss (FIGS. 4B and 8).

Rebalancing Wnt/Notch Signaling with LPAR5 Activation

Functional loss of a motor protein, Myosin Vb (MYO5B), induces a variety of deficits in intestinal epithelial function and causes a congenital diarrheal disorder, microvillus inclusion disease (MVID). Using the tamoxifen-induced epithelial-specific MYO5B knockout (Myo5bΔINT) mice and enteroid models, it has been reported that MYO5B loss disrupts progenitor cell differentiation. It was further shown that the administration of lysophosphatidic acid (LPA) can ameliorate epithelial cell differentiation and nutrient absorption. The present study investigated the mechanism of differentiation deficits, and compared the effect of selective agonists for LPAR subtypes. Selective agonists for LPAR1 and LPAR5, namely UCM-05194 and Compound-1, respectively, were synthesized. Adult Myo5bΔINT mice received UCM-05194, Compound-1, the commercially available LPAR2 agonist, GRI977143, or vehicle once a day for 4 days following after the tamoxifen injection. Mucosal histology and differentiated cell population were analyzed by immunostaining with whole slide imaging and digital image analysis tools. Transcription was analyzed by quantitative PCR with mRNA isolated from jejunal epithelial cells of mice. LPAR5 expression was significantly higher than other LPAR subtypes in both control and Myo5bΔINT-deficient intestines. While UCM-05194 or GRI977143 did not significantly alter the intestinal histology in the induced intestinal MYO5B-deficient mice, intraperitoneal injection of Compound-1 significantly improved villus/crypt ratio. Nutrient transporters of mature enterocytes, such as NHE3 and SGLT1 were mislocalized from apical membrane in MYO5B-deficient intestine and their localization were partially recovered to the apical membranes by Compound-1 treatment (FIGS. 2A-2E). Furthermore, the decrease in tuft cell numbers in mice with MYO5B loss was significantly increased by Compound-1 treatment, but not other LPAR agonists, suggesting that LPAR5 activation can ameliorate proper cell differentiation in MYO5B-deficient intestine. To identify the cellular mechanism of differentiation deficits, transcription for Wnt and Notch signaling molecules were assessed. Expression of epithelial-derived Wnt ligands were significantly downregulated by MYO5B loss, whereas Notch signaling mediators (DII1, DII4, Notch2, Hes1) were unchanged. Additionally, acute treatment with the Notch inhibitor, dibenzazepine (DBZ: 30 μmol/kg), restored the populations of secretory cells, suggesting that the Notch pathway is maintained in MYO5B-deficient intestine. Conclusion: MYO5B loss likely impairs progenitor cell differentiation in the small intestine partially mediated by Wnt/Notch imbalance. Re-balancing Wnt/Notch signaling with LPAR5 activation may represent an effective therapeutic approach for treatment of MVID.

### Example 2: Discussion

For treatment of chronic diarrhea and malabsorption, both inhibiting abnormal secretion as well as enhancing enterocyte maturation are important goals. Nevertheless, epithelial cell differentiation and maturation has not been well studied as a therapeutic target of diarrhea. It was recently shown that the disrupted epithelial cell differentiation and enterocyte maturation in MYO5B deficient intestine is likely another pathology that is underlying the malabsorption in MVID (Kaji et al., 2021; Kaji et al., 2020), in addition to MYO5B-mediated membrane trafficking defects shown in previous studies (Roland et al., 2011; Vogel et al., 2017). This study additionally presented that fatty acid oxidation (FAO) and mitochondrial function of intestinal epithelial cells were significantly impaired by functional MYO5B loss both in mouse tissues and enteroid models. Recent studies have revealed that FAO is critical for progenitor cell maintenance and differentiation, suggesting that MYO5B-mediated cellular metabolic activities are important for intestinal epithelial cells. Lysophosphatidic acid and the synthetic LPAR5 agonist, Compound-1 treatment similarly improved the expression patterns of mature enterocyte transporters, NHE3 and SGLT1, and the differentiation of tuft cell lineage in the Myo5bΔINT mouse model. Interestingly, LPAR5 activation robustly increased the ketogenic enzyme, Hmgcs2 expression in Myo5bΔINT mouse tissues that likely facilitated enterocyte and tuft cell maturation. This LPAR5-induced Hmgcs2 upregulation was not seen in control mice, suggesting that LPA target is specific in MYO5B-deficient epithelial cells. Daily Compound-1 treatment increased villus/crypt ratio by 150% in the small intestine of Myo5bΔINT mice, indicating that this drug is promising for treatment of malabsorption.

Despite those trophic effects of Compound-1, the Compound-1-treated Myo5bΔINT mice still lost body weight approximately 14% within 4 days in similar manner to that in vehicle-treated group. A predicted reason is that LPAR5-expressing vagal afferent nerves are activated by systemic Compound-1 administration and reduced e. Additional nutritional supplementation and/or fluid therapy will be needed to test in vivo. Subcutaneous tablet insertion was also tested to continuously deliver Compound-1. Unfortunately, low dose (0.2 mg/mouse) or high dose (2 mg/mouse) per tablet had no consistent effect on Myo5bΔINT mice, probably because of the fast degradation of Compound-1 in vivo. In wild type adult mice, Compound-1 was detected in plasma 1 hour after a bolus intraperitoneal injection (10 mg/kg) and disappeared (<10 ng/ml) within 6 hours. Compound-1 (C₂₀H₄₂NaO₅PS, mw: 448.57) is designed as metabolically stabilized LPA analogue, and its EC₅₀ on human LPAR5 shows higher potency (0.26 nM) than natural LPA(18:1) (55 nM) (Jiang et al., 2013). Further analysis of Compound-1 kinetics in human cell culture system is required to determine clinical dosage.

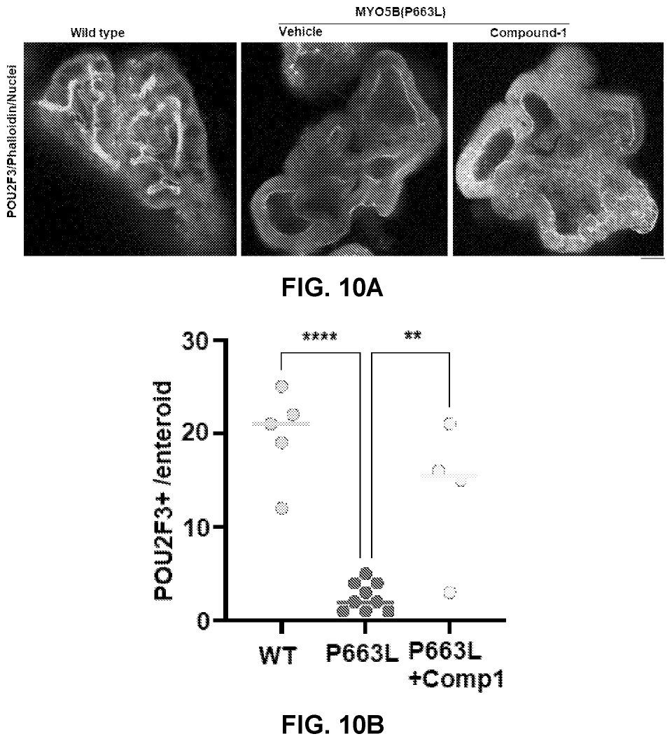

Direct MYO5B function in intestinal progenitor cells has not been well characterized. In the present study, the enteroid models from MVID patient-mimicking mouse (Myo5b(G519R)) and pig (MYO5B(P663L)) demonstrated tuft cell differentiation defect that is similar to MYO5B deficient mouse (Myo5bΔINT) model. In both mouse enteroid models lacking MYO5B function, decreased metabolic enzymes and imbalance of Wnt/Notch signaling was identified that alter cell lineage differentiation. Other interesting transcriptional signature was the upregulation of cell adhesion and cytoskeleton molecules that also indicate disrupted cell differentiation by MYO5B loss. Together with the morphological changes of epithelial mitochondria (FIG. 7), these transcription changes suggest that MYO5B is required to maintain proper energy metabolism and lineage differentiation of progenitor cells. Compound-1 treatment significantly (log 2 fold change >1, adjusted p<0.05) altered 85 genes in Myo5bΔINT enteroids, and 21 genes were significantly changed by MYO5B loss compared to control enteroids. Highlighted genes, which were MYO5B loss-increased and Compound-1-reversed, were implicated in cellular stress responses induced by inflammation or infection (FIG. 4B). This result indicates that trophic effect of Compound-1 treatment that was seen in vivo was partly mediated by direct Compound-1 effect on stressed epithelial cells. Furthermore, Compound-1 significantly improved tuft cell differentiation, which can be an indicator of epithelial maturation, in MYO5B(P663L) pig enteroids (FIGS. 10A-10B).

In conclusion, Compound-1 treatment has intestinal trophic effects, bypassing the deficits induced by inactivation of MYO5B, via cell-autonomous pathways that may be mediated by progenitor cell metabolism and proper differentiation toward absorptive enterocytes and sensory tuft cells, resulting in increasing nutrient absorption (FIG. 5).

Patient enteroid cultures were established in a collaboration with the PediCODE Consortium to develop a drug screening system. MVID enteroids were generated from patient biopsies with the homozygous MYO5B(P660L) mutation or early truncation of MYO5B (knockout), and as well as age-matched infant enteroids (unpublished data). Compound-1 treatment will be tested on these MVID patient enteroids to investigate its efficacy on actual patient cells and to determine the best dose. The deficits of epithelial energy metabolism associated with differentiation disruption might be a common mechanism between MVID and other chronic diarrheal diseases. The effect of Compound-1 on other congenital diseases was also studied, including DGAT1 deficiency and tufting enteropathy, in which enteroid cultures are now shared with PediCODE members.

Compound-1 has an agonistic effect on human LPAR3 (Jiang et al. 2013), which is not predominant in intestinal epithelial cells. Since LPAR5 and LPAR3 are expressed in non-epithelial tissues, such as immune cells and brain, long-term effect of Compound-1 treatment needs to consider off-target effects. In the future study, the interaction between MYO5B deficient epithelial cells and mucosal immune cell populations should be addressed. In the RNA-sequencing data of both tissue epithelial cells and enteroid cultures, important chemokine ligands, Ccl9 and Ccl25, were significantly downregulated in MYO5B deficient epithelial cells. Ccl9/Ccl25 produced in enterocytes are required for the activation of gut-associated lymphoid tissues (GALT) that support epithelial barrier function. The abnormality of GALT has not been investigated in MVID patients, but immune deficiency must be considered in long-term care.

### Example 3: Materials and Methods

### Mice

All animal studies were performed with approval from the Institutional Animal Care and Use Committee of Vanderbilt University Medical Center (M2000104). At the 8-10 week of age, Myo5bΔINT mice and littermate controls (VilCre^{ERT2}; Myo5b^{+/flox} and Myo5b^{flox/flox}) received a single dose of tamoxifen (100 mg/kg) by intraperitoneal (IP) injection (day 0) (Weis et al.). LPA (3 mg/kg, 18:1 1-oleoyl-Lyso PA, 857231-P, Avanti Polar Lipids) solution was prepared and administered by intraperitoneal (ip) injection once a day for 4 days as previously reported (Kaji et al., 2020). Compound-1 was dissolved in PBS, and UCM-05194 was dissolved in Captisol solution. Body weight changes and diarrhea symptoms were investigated daily. On day 4, mice were euthanized and the duodenum (0-8 cm from the pyloric ring), jejunum (8 cm following the duodenum), ileum (distal 8 cm from the ileocecal junction), and colon were collected for pathological assessment as previously reported (Kaji et al.).

In Vitro Induced MYO5B-Knockout (iKO) Enteroids

Enteroids were generated from jejunal crypts of adult VilCre^{ERT2}; Myo5b^{flox/flox} and VilCre^{ERT2}; Myo5b^{flox/G519R} mice (Burman & Momoh CMGH) without tamoxifen treatment and passaged twice with mechanical breakdown. One day after the second passage, 1 μM 4-OH-tamoxifen (SML1666, Sigma) was added into the IntestiCult Organoid Growth Medium (Mouse, Stem Cell Technology) and incubated for 24 hours to induce Cre recombinase. After the incubation with 4-OH-tamoxifen (induced KO or G519R) or the vehicle, 100% ethanol (Control), the medium was replaced with the modified Minigut medium (Mahe et al., 2013) containing 5% Noggin conditioned medium and 5% R-spondin conditioned medium (Vanderbilt Organoid Core) to withdraw Wnt and EGF for enhancing cell differentiation (Kaji JCI Insight). Some iKO enteroids were incubated with 100 nM Compound-1 for 2 days in differentiation conditions.

### RNA Extraction and Sequencing

Following differentiation, enteroids were placed in Organoid Harvesting Solution (3700-100-01; Cultrex) for 1 hr at 4° C. Enteroids were washed with PBS and centrifuged. Enteroid pellets were immersed in TRIzol reagent (15596026; Invitrogen) supplemented with glycogen (G1767; Sigma Aldrich) and stored at −20° C. Total RNA was extracted from enteroids following the manufacturer's instructions. RNA-sequencing with 4 samples per group and differential expressing gene analysis were performed in Novogene.

### Tissue Immunostaining

Immunofluorescence staining and imaging were performed as previously reported (Kaji, JCI Insight). Four-μm-thin sections were deparaffinized and rehydrated before performing antigen retrieval using 10 mM sodium citrate buffer, contained 0.05% Tween 20 (pH 6), in a pressure cooker for 15 minutes. After cooling down on ice, slides were rinsed in PBS and blocked with protein block serum-free (Dako) for 1 hour at r/t or overnight at 4° C. The primary antibodies against NHE3 (1:200), SGLT1 (0.5 μg/ml) (Kaji et al., 2020), DCLK1 (AF647-conjugated, 1:2000), KRT19 (1:250), CD3, HMGCS2 (1:50), or Actg1 (1:100) were diluted in Dako diluent in background reducing compound (S3022) and incubated on sections overnight at 4° C. After rinsing in 0.1 M PBS, the slides were incubated with the corresponding secondary antibodies conjugated with fluorescence (Jackson Laboratory) for 1 hour. Slides were rinsed in 0.1 M PBS and counterstained with Hoechst 33342 (4 mM, Thermo Fisher Scientific, Waltham, MA) in PBS. The stained slides were cover-slipped with ProLong Gold Antifade Reagent (P36934, Thermo Fisher). Whole slide images were scanned by a Versa imager 200 with a 20× objective (Leica Biosystems, Buffalo Grove, IL) in the Vanderbilt Digital Histology Shared Resource (DHSR). Some immunofluorescence images were taken by using a Zeiss Axio Imager M2 with ApoTome (Carl Zeiss Microscopy, LLC, White Plains NY).

### Pig MVID Model Enteroids

Pig MVID enteroids that possess a homozygous mutation at MYO5B(P663L) or wild type MYO5B were established from jejunal crypts as previously reported (Engevik 2020). Pig enteroids were expanded in Matrigel (Corning) immersed in IntestiCult Organoid Growth Medium (Human, OGM, Stem Cell Technology). Organoid growth was assessed in 96-well plate and continuously imaged whole wells using a JuLi™ Stage (NanoEn Tek Inc., Waltham, MA). Organoid perimeter was measured by using FIJI, and organoid forming rate was calculated as sphere numbers per plated cell numbers 7 days after the passaging. N=7-9 wells from each genotype. In a different set of culture, tuft cells were differentiated in Organoid Differentiation Medium (ODM, Stem Cell Technology) for 5 days, following after 2 days culture in OGM after passaging. MYO5B(P663L) enteroids were incubated with 100 nM Compound-1 or vehicle (PBS) in ODM.

### Whole-Mount Organoid Staining

Organoids were fixed with 10% NBF for 30 min in Matrigel domes, rinsed with cold PBS containing 0.1% FBS and centrifuged to remove Matrigel. Fixed organoids were blocked with 5% normal donkey serum in PBS containing 0.3% Triton X-100 for 2 hours and incubated with the pGirdin (1:100) or POU2F3 (1:200) antibody diluted with the blocking solution for 2 days at 4° C. After rinsing in PBS with Triton X-100, donkey anti-rabbit Cy3 antibody (2.5 μg/ml), Alexa Fluor 488 phalloidin (1:400, A12379, Thermo Fisher), and Hoechst 33342 (2 mM) were applied for 2 hr. Whole organoids images were taken by a Nikon Ti-E microscope with an A1R laser scanning confocal system (Nikon Instruments Inc., Melville, NY).

### Electron Microscopy

After euthanasia, mouse tissues were fixed with 2.5% glutaraldehyde in 0.1 M cacodylate by trans-cardiac perfusion following warm PBS perfusion. Small pieces of intestine were further fixed in the same fixative, followed by sequential post-fixation in 1% tannic acid, 1% OsO₄, and en bloc stained in 1% uranyl acetate. All samples were dehydrated using a graded ethanol series. TEM samples were subsequently infiltrated with Epon-812 using propylene oxide as the transition solvent, followed by polymerization at 60° C. for 48 hours. Samples were sectioned at a nominal thickness of 70 nm using a Leica UC7 ultramicrotome. TEM imaging was performed using a Tecnai T12 operating at 100 keV using an AMT nanosprint CMOS camera.

### Digital Image Analysis (DIA)

The DIA code to quantify the tuft cell populations was developed at the DHSR on Python platform as described previously (Kaji 2021).

### Statistics

Results in graphs are expressed as Mean±S.D., Statistical differences were determined with a significant P value of <0.05 using GraphPad Prism 9 statistical software. The test used in each analysis is described in the Brief Description of the Drawings.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Bowman, D. M., et al. (2022). Altered MYO5B Function Underlies Microvillus Inclusion Disease: Opportunities for Intervention at a Cellular Level. Cell Mol Gastroenterol Hepatol 14, 553-565.
2. Burman, A. et al. (2022). Modeling of a Novel Patient-Based MYO5B Point Mutation Reveals Insights Into MVID Pathogenesis. Cell Mol Gastroenterol Hepatol.
3. Chen, L., et al. (2019). HNF4 Regulates Fatty Acid Oxidation and Is Required for Renewal of Intestinal Stem Cells in Mice. Gastroenterology, S0016-5085 (0019) 41587-41584.
4. Cheng, C. W., et al. (2019). Ketone Body Signaling Mediates Intestinal Stem Cell Homeostasis and Adaptation to Diet. Cell 178, 1115-1131.e1115.
5. Cutz, E., et al. (1989). Microvillus inclusion disease: an inherited defect of brush-border assembly and differentiation. N Engl J Med 320, 646-651.
6. Davidson, G. P., et al. (1978). Familial enteropathy: a syndrome of protracted diarrhea from birth, failure to thrive, and hypoplastic villus atrophy. Gastroenterology 75, 783-790.
7. Engevik, A. C., et al. (2020). Editing Myosin VB Gene to Create Porcine Model of Microvillus Inclusion Disease, With Microvillus-Lined Inclusions and Alterations in Sodium Transporters. Gastroenterology 158, 2236-2249.e2239.
8. Erickson, R. P., et al. (2008). Navajo microvillous inclusion disease is due to a mutation in MYO5B. Am J Med Genet A 146a, 3117-3119. 10.1002/ajmg.a.32605.
9. González-Gil, I., et al. (2020). A Novel Agonist of the Type 1 Lysophosphatidic Acid Receptor (LPA (1)), UCM-05194, Shows Efficacy in Neuropathic Pain Amelioration. J Med Chem 63, 2372-2390.
10. Haber, A. L., et al. (2017). A single-cell survey of the small intestinal epithelium. Nature 551, 333-339.
11. Jenkin, K. A., et al. (2018). Expression of lysophosphatidic acid receptor 5 is necessary for the regulation of intestinal Na(+)/H(+) exchanger 3 by lysophosphatidic acid in vivo. Am J Physiol Gastrointest Liver Physiol 315, G433-9442.
12. Jiang, G., et al. (2013). Phosphorothioate analogs of sn-2 radyl lysophosphatidic acid (LPA): metabolically stabilized LPA receptor agonists. Bioorg Med Chem Lett 23, 1865-1869.
13. Kaji, I., et al. (2020). Lysophosphatidic Acid Increases Maturation of Brush Borders and SGLT1 Activity in MYO5B-deficient Mice, a Model of Microvillus Inclusion Disease. Gastroenterology.
14. Kaji, I., et al. (2021). Cell differentiation is disrupted by MYO5B loss through Wnt/Notch imbalance. JCI Insight 6.
15. Konno, T., et al. (2019). Role of lysophosphatidic acid in proliferation and differentiation of intestinal epithelial cells. PLOS One 14, e0215255.
16. Li, C., et al. (2005). Lysophosphatidic acid inhibits cholera toxin-induced secretory diarrhea through CFTR-dependent protein interactions. J Exp Med 202, 975-986.
17. Liang, Z., et al. (2022). Compensatory Upregulation of LPA(2) and Activation of the PI3K-Akt Pathway Prevent LPA (5)-Dependent Loss of Intestinal Epithelial Cells in Intestinal Organoids. Cells 11, 2243.
18. Liang, Z., et al. (2022). Survival of Stem Cells and Progenitors in the Intestine Is Regulated by LPA<sub>5</sub>-Dependent Signaling. Cell Mol Gastroenterol Hepatol 14, 129-150.
19. Lin, S., et al. (2010). Lysophosphatidic acid stimulates the intestinal brush border Na(+)/H(+) exchanger 3 and fluid absorption via LPA(5) and NHERF2. Gastroenterology 138, 649-658.
20. Liu, W., et al. (2021). The development of modulators for lysophosphatidic acid receptors: A comprehensive review. Bioorg Chem 117, 105386.
21. Mahe, M. M., et al. (2013). Establishment of Gastrointestinal Epithelial Organoids. Curr Protoc Mouse Biol 3, 217-240.
22. Meduri, B., et al. (2021). Lysophosphatidic acid (LPA) receptor modulators: Structural features and recent development. Eur J Med Chem 222, 113574.
23. Muller, T., et al. (2008). MYO5B mutations cause microvillus inclusion disease and disrupt epithelial cell polarity. Nat Genet 40, 1163-1165.
24. Roland, J. T., et al. (2011). Rab GTPase-Myo5B complexes control membrane recycling and epithelial polarization. Proc Natl Acad Sci USA 108, 2789-2794.
25. Ruemmele, F. M., et al. (2006). Microvillous inclusion disease (microvillous atrophy). Orphanet J Rare Dis 1, 22.
26. Stine, R. R., et al. (2019). PRDM16 Maintains Homeostasis of the Intestinal Epithelium by Controlling Region-Specific Metabolism. Cell Stem Cell 25, 830-845.e838.
27. Thiagarajah, J. R., et al. (2018). Advances in Evaluation of Chronic Diarrhea in Infants. Gastroenterology 154, 2045-2059.e2046.
28. Vogel, G. F., et al. (2017). Abnormal Rab11-Rab8-vesicles cluster in enterocytes of patients with microvillus inclusion disease. Traffic 18, 453-464.
29. Weis, V. G., et al. (2016). Loss of MYO5B in mice recapitulates Microvillus Inclusion Disease and reveals an apical trafficking pathway distinct to neonatal duodenum. Cell Mol Gastroenterol Hepatol 2, 131-157.

What is claimed is:

1. A method of treating or preventing a disease in a subject, wherein the disease is associated with a mutation in Myosin Vb (MYO5B), a mutation in UNC45A, a mutation in syntaxin 3, decreased lysophosphatidic acid receptor 5 (LPAR5) expression, or any combination thereof, and wherein the method comprises administering a composition comprising a therapeutically effective amount of an LPAR5 agonist to the subject.

2. The method of claim 1, wherein the LPAR5 agonist comprises one or more of lysophosphatidic acid, [(2S)-2-hydroxy-3-octadecoxypropyl] dihydrogen phosphate, [(2Z,6Z)-3,7,11-trimethyldodeca-2,6,10-trienyl] dihydrogen phosphate, farnesyl diphosphate, octyl thiophosphatidic acid, N-arachidonoylglycine, or a compound having Formula I or a derivative thereof:

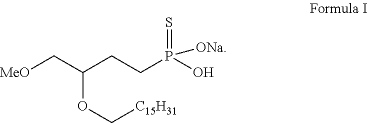

3. The method of claim 1, wherein the subject is a mammal.

4. The method of claim 3, wherein the mammal is a human, pig, mouse, rat, dog, cat, or rabbit.

5. The method of claim 1, wherein the disease comprises microvillus inclusion disease (MVID), cholestatic liver disease, diacylglycerol-acyltransferase 1 (DGAT1) deficiency, congenital tufting enteropathy (CTE), another tufting enteropathy, variant MVID, an MVID-like phenotype, or any combination thereof.

6. The method of claim 5, wherein the disease is MVID.

7. The method of claim 1, wherein the mutation comprises a truncated protein, an insertion, a deletion, a misfolded protein, or any combination thereof.

8. The method of claim 1, wherein the mutation comprises a homozygous mutation or a compound heterozygous mutation.

9. The method of claim 1, wherein the composition further comprises at least one excipient.

10. The method of claim 1, wherein treating the disease reduces severity of at least one symptom of the disease in the subject compared to the symptom prior to initiating treatment.

11. The method of claim 10, wherein the symptom comprises diarrhea, malabsorption, or any combination thereof.

12. The method of claim 1 wherein administering comprises subcutaneous administration, intravenous administration, transdermal administration, or oral administration.

13. The method of claim 1, wherein the composition is administered one or more times per day for at least one month.

14. The method of claim 1 wherein the composition is administered one or more times per day for at least one year.

15. The method of claim 1, wherein the composition is administered at a dosage of from about 5 mg to about 10 mg per kg of body weight of the subject.

16. The method of claim 1, further comprising administering at least one additional treatment to the subject.

17. The method of claim 16, wherein the at least one additional treatment comprises total parenteral nutrition.

18. A kit for treating a disorder associated with a mutation in Myosin Vb (MYO5B), a mutation in UNC45A, a mutation in syntaxin 3, decreased lysophosphatidic acid receptor 5 (LPAR5) expression, or any combination thereof in a subject, the kit comprising:
(a) a therapeutically effective amount of a compound of Formula I and at least one pharmaceutically acceptable excipient; and 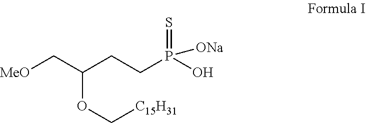
(b) instructions for using the kit.

19. The kit of claim 18, wherein the compound of Formula I and the at least one pharmaceutically acceptable excipient are packaged separately.

20. The kit of claim 18, wherein the therapeutically effective amount of the compound comprises a unit dosage of from about 12.5 mg to about 1000 mg.
